(19) United States
(12) Patent Application Publication
(10) Pub. No.: US 2024/0299244 A1
(43) Pub. Date: Sep. 12, 2024

(54) AUTOMATIC NASAL WASHER

(71) Applicant: Shenzhen Shufang Innovation Technology Co., Ltd., Shenzhen (CN)

(72) Inventor: Hejiang Ma, Hunan (CN)

(21) Appl. No.: 18/185,585

(22) Filed: Mar. 17, 2023

(30) Foreign Application Priority Data
Mar. 7, 2023 (CN) 202310212214.4

(51) Int. Cl. A61H 35/04

(52) U.S. Cl. CPC A61H 35/04; A61H 2205/023

(57) ABSTRACT

An automatic nasal washer includes a water storage portion, a control portion, and a nasal docking portion. The water storage portion is arranged with a water storage chamber. The control portion is fixed to the water storage portion and is arranged with: a water inlet channel communicated with the water storage chamber; a switch valve configured to control the water inlet channel to be communicated with or dis-communicated from the water storage chamber; a water return channel communicated with the water inlet channel; and a suction member configured to provide a negative pressure to the water return channel. The nasal docking portion is connected to a point where the water inlet channel is connected to the water return channel; and configured to output washing water from the water inlet channel to a nasal cavity and to withdraw waste water flowing from the nasal cavity to the water return channel.

### CROSS REFERENCE TO RELATED APPLICATIONS

The present disclosure claims the priority of Chinese patent application No. CN202310212214.4, filed on Mar. 7, 2023, and the contents of which are hereby incorporated in its entirety.

### TECHNICAL FIELD

The present disclosure relates to the field of electronic elements, and in particular to an automatic nasal washer.

### BACKGROUND

Currently, a nasal washer may be pressed manually to provide a power to drive water to flow and clean a nasal cavity of a user. However, in this way, the nasal washer needs to be connected to an external water source and an external water return device. Operations performed by the user may be complicated and may be time consuming.

### SUMMARY OF THE DISCLOSURE

The present disclosure provides an automatic nasal washer, which can be used easily.

The present disclosure provides an automatic nasal washer, including a water storage portion, a control portion, and a nasal docking portion. The water storage portion is arranged with a water storage chamber. The control portion is fixed to the water storage portion and is arranged with: a water inlet channel communicated with the water storage chamber; a switch valve configured to control the water inlet channel to be communicated with or dis-communicated from the water storage chamber; a water return channel communicated with the water inlet channel; and a suction member configured to provide a negative pressure to the water return channel. The nasal docking portion is connected to a point where the water inlet channel is connected to the water return channel; and is configured to output washing water from the water inlet channel to a nasal cavity of a user and to withdraw waste water flowing from the nasal cavity to the water return channel.

In some embodiments, the water storage portion includes a storage housing, a filter housing, and a cover. The water storage chamber is defined in the storage housing, the water storage chamber has a top opening and a bottom opening, the cover closes the top opening. The filter housing is received inside the water storage chamber and is connected to the top opening. The control portion is connected to the bottom opening, and the switch valve is disposed at the bottom opening.

In some embodiments, the control portion includes a central control housing, a power transmission member, a valve drive member, and a control button. The central control housing is sealed to the bottom opening and defines a water inlet port communicated with the bottom opening; the switch valve is disposed at the water inlet port; the water inlet channel and the water return channel are arranged inside the central control housing; the control button is configured to control the switch valve to be opened or closed; the control button is pressable and arranged on a side of the central control housing; the valve drive member is connected to the switch valve to provide an abutting force to close the valve; the power transmission member is connected to the control button and the valve drive member to transfer a pressing force of the control button to the valve drive member to control the valve drive member to open the switch valve.

In some embodiments, the control portion further includes a suction trigger connected with the control button. The suction trigger is received inside the central control housing and is disposed out of the water inlet channel and the water return channel; the suction member is received in the central control housing and is disposed out of the water inlet channel and the water return channel, the suction member is electrically connected to the suction trigger; the suction trigger is configured to control the suction member to start operating or stop operating; and while the control button is controlling the switch valve to be opened via the power transmission member, the suction trigger is being triggered to control the suction member to start operating.

In some embodiments, the automatic nasal washer further includes a water return portion. The water return section is fixed on a side of the control portion away from the water storage portion and defines a waste water chamber communicated with the water return channel, the waste water chamber is configured to receive the waste water flowing from the water return channel.

In some embodiments, the control portion further includes a battery, a charging interface, and a circuit board. The battery is fixedly arranged inside the central control housing; the circuit board is fixedly arranged inside the central control housing and electrically connected to the battery, the suction member, and the suction trigger; the charging interface is arranged on a side of the central control housing and electrically connected to the circuit board to allow an external power supply to charge the battery.

In some embodiments, the control portion further includes a status indicator light, the status indicator light is arranged on a side of the central control housing and electrically connected to the circuit board to indicate a charging state of the battery.

In some embodiments, the control portion is arranged with a gyroscope and a controller; the gyroscope is configured to sense an attitude of the automatic nasal washer and is electrically connected to the controller to send the attitude of the automatic nasal washer to the controller; the controller is electrically connected to the suction member and is configured to control the suction member to stop operating in response to the attitude of the automatic nasal washer being inclined.

In some embodiments, the nasal docking portion is arranged with a first port and a second port side by side with the first port; an end of the first port is connected to the water inlet channel, an end of the second port is connected to the water return channel; the other end of the first port and the other end of the second port are connected to two nostrils of the user, respectively.

In some embodiments, the nasal docking portion is arranged with a diversion head communicated with the water inlet channel and the water return channel; and the diversion head is rotatably arranged on the control portion to change a direction of the first port and the second port.

According to the present disclosure, the control portion of the nasal washer may control a switch valve to be open or close to automatically allow the nasal docking portion to output cleaning water to the nasal cavity of the user and to allow the nasal docking portion to automatically withdraw waste water from the nasal cavity of the user. Operations performed by the user may be simple and quickly.

### BRIEF DESCRIPTION OF THE DRAWINGS

In order to illustrate the technical solutions of the embodiments of the present disclosure more clearly, the accompanying drawings used in the embodiments will be briefly described in the following. Apparently, the drawings in the following description are only some of the embodiments of the present disclosure. Any ordinary skilled person in the art may obtain other drawings based on these drawings without any creative work.

FIG. 1 is a perspective view of an automatic nasal washer according to an embodiment of the present disclosure.

FIG. 2 is a cross-sectional view of an automatic nasal washer according to an embodiment of the present disclosure.

FIG. 3 is an exploded view of an automatic nasal washer according to an embodiment of the present disclosure.

FIG. 4 is a schematic view of a control module of an automatic nasal washer according to an embodiment of the present disclosure.

FIG. 5 is another cross-sectional view of an automatic nasal washer according to an embodiment of the present disclosure.

### DETAILED DESCRIPTION

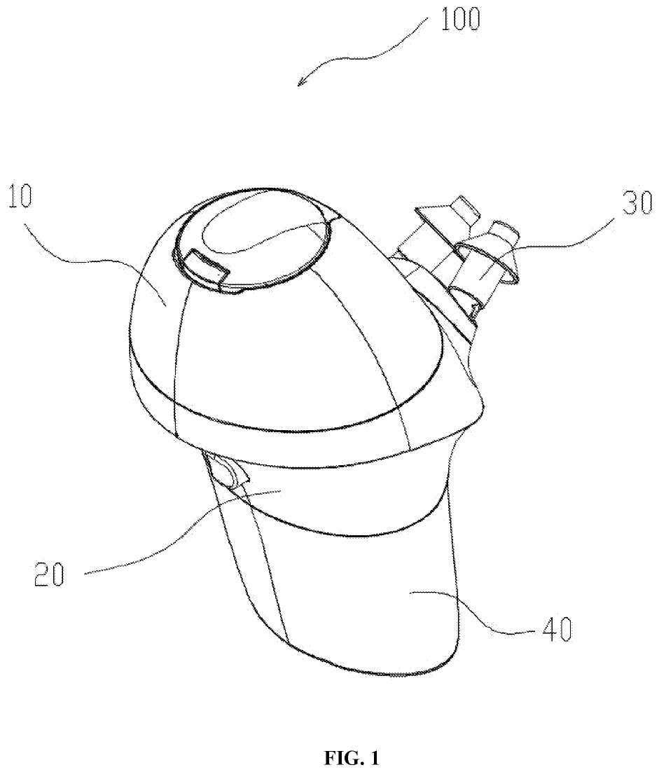

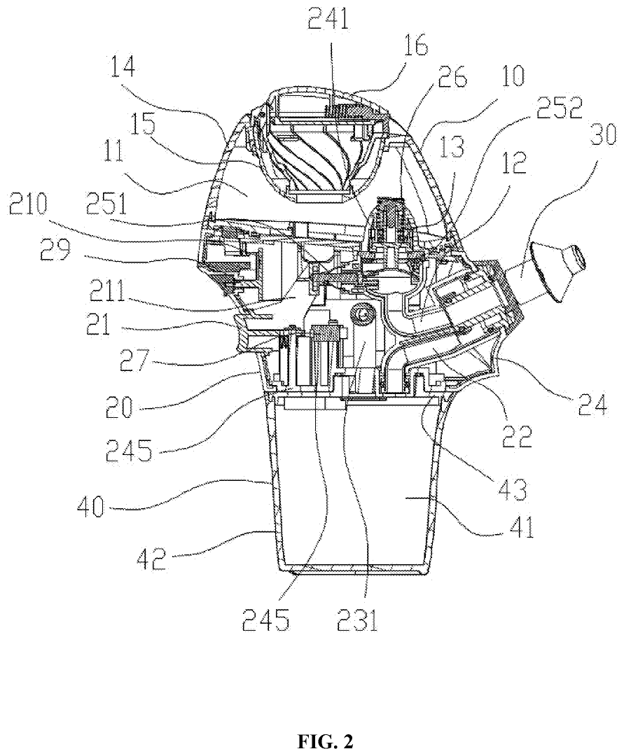

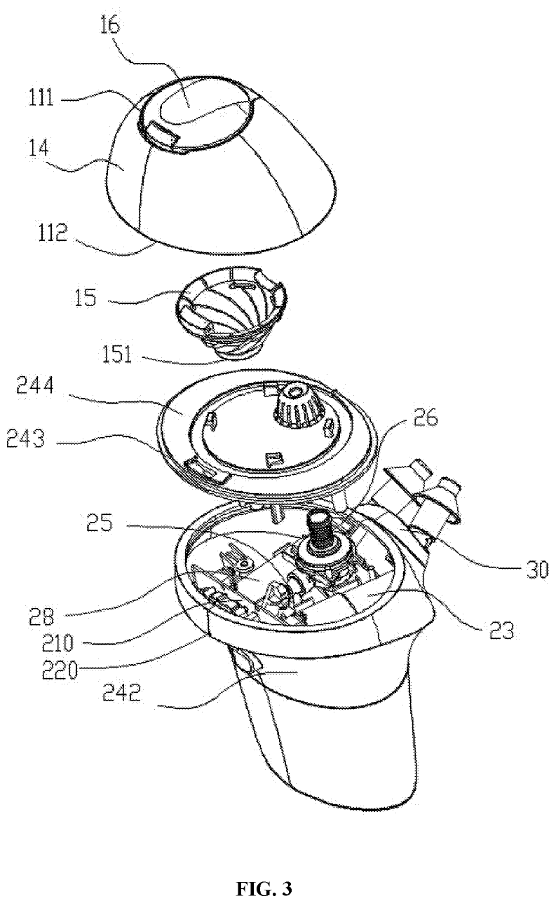

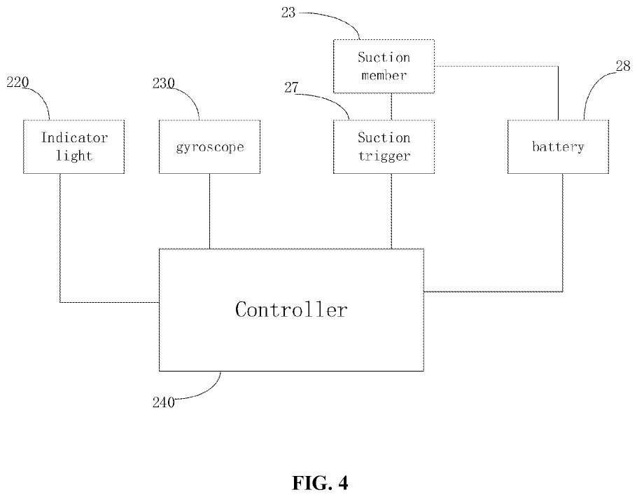

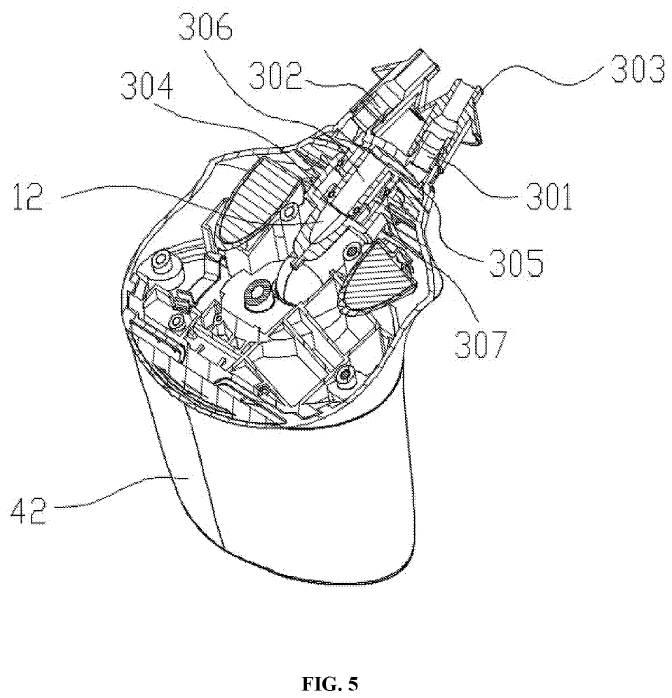

As shown in FIG. 1 to FIG. 5, the present disclosure provides an automatic nasal washer 100. The automatic nasal washer 100 includes a water storage portion 10, a control portion 20, a nasal docking portion 30 and a water return portion 40. The water storage portion 10 has a water storage chamber 11. The control portion 20 is fixed to the water storage portion 10. The control portion 20 has a water inlet channel 12 communicating with the water storage chamber 11 and is arranged with a switch valve 13 configured to control the water inlet channel 12 to be communicated with or dis-communicated from the water storage chamber 11. The control portion 20 is further arranged with a control button 21 configured to control the switch valve 13 to be open or close. The control portion 20 further has a water return channel 13 communicated with the water inlet water 12 and is arranged with a suction member 23 configured to provide a negative pressure to the water return channel 22. The nasal docking portion 30 is docked to a point where the water inlet channel 12 is communicated with the water return channel 22. The nasal docking portion 30 is configured to output washing water from the water inlet channel 12 to a nasal cavity of a user and configured to withdraw waste water from the nasal cavity of the user to the water return channel 22. The water return portion 40 is fixed to a side of the control portion 20 away from the water storage portion 10 and has a waste water chamber 41 communicated to the water return channel 22. The waste water chamber 41 is configured to receive the waste water flowing from the water return channel 22.

In the present disclosure, the switch valve 13 may be controlled to be open or close by the control portion 20 to automatically allow the nasal docking portion 30 to output the washing water to the nasal cavity of the user and to allow the nasal docking portion 30 to automatically withdraw the waste water from the nasal cavity of the user. Operations performed by the user may be simple and quickly.

In the present embodiment, the water storage chamber 11 is defined to store the nasal cleansing solution. The nasal cleansing solution may be saline, a cleansing solution, or pure water. A top of the water storage chamber 11 is configured to receive water from the outside. A bottom of the water storage chamber 11 is connected to the switch valve 13. When the switch valve 13 is opened, the nasal cleaning solution may enter the water inlet channel 12 quickly due to the gravity. The gravity and a suction force generated by the suction member 23 may cooperatively allow the nasal cleaning solution to flow more quickly, improving a cleaning efficiency.

In the present embodiment, the control portion 20 is fixed to a bottom of the water storage portion 10. The control portion 20 is sealed to the water storage portion 10, effectively preventing the nasal cleansing solution from leaking out of the water storage chamber 11. The control button 21 is disposed on a surface of the control portion 20. The control button 21 can be pressed for operating. When the control button 21 is pressed, the switch valve 13 is opened, and at the same time, the suction member 23 operates. The suction members 23 provides the negative pressure to the water return channel 22. In this way, the water inlet channel 12 is enabled to take the nasal cleansing solution from the water storage chamber. In this way, the user does not need to provide an additional driving pressure to the nasal cleansing solution, the automatic nasal washer does not need to be inverted upside down, and the gravity is not required to drive the nasal cleansing solution to flow.

In the present embodiment, the nasal docking portion 30 is sealed to the water inlet channel 12 and the water return channel 22. The nasal docking portion 30 is disposed at a end of the water inlet channel 12 away from the switch valve 13 and disposed at an end of the water return channel 22 away from the waste water chamber 41. When the suction member 23 applies the negative pressure to the water return channel 22, the nasal docking portion 30 is sealed to the nasal cavity of the user, such that the negative pressure is generated at the water inlet channel 12. At this time point, the switch valve 13 is open, the nasal cleansing solution in the water storage chamber 11 is driven by the negative pressure to flow rapidly into the water inlet channel 12.

The nasal cleansing solution washes flows through the water inlet channel 12 to wash the nasal cavity of the user. Eventually, the nasal cleansing solution is driven by the negative pressure to flow from the nasal cavity to the water return channel 22. The water return channel 22 guides the waste water, which is obtained after cleaning the nasal, to the waste water chamber 41. Nasal cleaning is completed.

In the present embodiment, the water return portion 40 is fixed to the bottom of the control portion 20. The water return portion 40 is detachably connected to the control portion 20. The control portion 20 may seal an opening of the water return portion 40 disposed at the top of the waste water chamber 41. When the control portion 20 is detached from the water return portion 40, the opening of the waste water chamber 41 is opened, and the waste water may be removed from the opening of the waste water chamber 41.

Further, the water storage portion 10 includes a storage housing 14, a filter housing 15, and a cover 16. The water storage chamber 11 is arranged inside the storage housing 14. The water storage chamber 11 has a top opening 111 and a bottom opening 112. The cover 16 closes the top opening 111. The filter housing 15 is disposed inside the water storage chamber 11 and is connected to the top opening 111. The control portion 20 is aligned to and connected to the bottom opening 112. The switch valve 13 is disposed at the bottom opening 112.

In the present embodiment, the filter housing 15 is funnel shaped. An end of the filter housing 15 having a larger opening is aligned to and connected to the top opening 111. An end of the filter housing 15 having a smaller opening is received in the water storage chamber 11. An inner side of the filter housing 15 defines a plurality of vortex fluid guiding slots extending from the end having the larger opening to the end having the smaller opening. The plurality of vortex fluid guiding slots may guide the nasal cleansing solution to flow rapidly to the bottom opening 112 and may drive the solution in the water storage chamber 11 to rotate, enabling water and the nasal cleansing salt in the water storage chamber 11 to be mixed well. It shall be understood that when the user uses the automatic nasal washer 100 to clean the nasal cavity, water may firstly be added to the water storage chamber 11, and nasal cleansing salt may be added to the water storage chamber 11 subsequently. Further, the nasal cleansing salt is mixed with and dissolved in the water in the water storage chamber 11, such that the nasal cleansing solution is prepared. The end of the filter housing 15 having the smaller opening is further arranged with a filter 151. The filter 151 is configured to filter any nasal cleansing salt that is not fully dissolved out of the nasal cleansing solution, preventing the undissolved nasal cleansing salt from flowing directly into the nasal cavity of the user, and preventing discomfort during the cleansing. The end of the filter housing 15 having the larger opening is partially exposed out of the top opening 111. Further, the end of the filter housing 15 having the larger opening is securely connected to and sealed to an edge of the top opening 111, ensuring stability of the filter housing 15. The part of the end of the filter housing 15 having the larger opening exposed out of the top opening 111 is rotatably connected to the cover 16, allowing the cover 16 to rotate with respect to the storage housing 14 to expose or to cover the water storage chamber. The cover 16 is further arranged with a buckle configured to provide a closing force to allow the cover 16 to be locked tightly with the filter housing 15 or configured to provide an unlocking force to allow the cover 16 to be unlocked with the filter housing 15. The buckle may be pressed to allow the buckle to be detached from a socket of the filter housing 15. The buckle may be detached from the cover 16 relative to the filter housing 15. In this way, the cover 16 may open the end of the filter housing 15 having the larger opening and the top opening 111. When the buckle is inserted in the socket of the filter housing 15, the cover 16 closes the end of the filter housing 15 having the larger opening and the top opening 111.

In the present embodiment, the switch valve 13 is disposed at an inside of the bottom opening 112. The switch valve 13 is misaligned with the end of the filter housing 15 having the smaller opening, preventing the nasal cleansing solution in the filter housing 15 from flowing directly to the switch valve 13. A scale line is arranged at an outside of the storage housing 14. The storage housing 14 may be a transparent housing, allowing the user to easily observe how much water is stored inside the water storage housing 14.

Furthermore, the control portion 20 includes a central control housing 24, a power transmission member 25, and a valve drive member 26. The central control housing 24 is sealed to the bottom opening 112 and defines a water inlet port 241 aligned to and communicated with the bottom opening 112. The switch valve 13 is disposed at the water inlet port 241. The water inlet channel 12 and the water return channel 22 are disposed at an inside of the central control housing 24. The control button 21 is pressable and disposed on a side of the central control housing 24. The valve drive member 26 is connected to the switch valve 13 to provide an abutting force to close the valve. The power transmission member 25 is connected to the control button 21 and the valve drive member 26 to transmit a pressing force of the control button 21 to the valve drive member 26 to control the valve drive member 26 to open the switch valve 13.

In the present embodiment, the central housing 24 includes a side housing 242 and a top cover 243. The top cover 243 is fixed to an end of the side housing 242 facing towards the storage housing 14. The top cover 243 seals the bottom opening 112. Specifically, a sealing member 244 is arranged between the top cover 243 and the storage housing 14. The top cover 243 is fixedly connected to the storage housing 14 via the sealing member 244. The sealing member 244 is arranged inside the water storage chamber 11 and is arranged with an inclined face that guides the nasal cleansing solution to flow to the switch valve 13. A top edge of the side housing 242 is aligned to and sealed to an edge of the bottom opening 112. The top cover 243 is arranged with a protrusion extending to the inside of the water storage chamber 11. The protrusion defines the water inlet port 241 and a moving cavity communicated with the water inlet port 241. An end of the water inlet channel 12 is aligned to and communicated with the moving cavity. The switch valve 13 is movable between the moving cavity and an opening of the water inlet channel 12. The valve drive member 26 is a spring. The valve drive member 26 is operating between the switch valve 13 and an inner wall of the protrusion to provide an elastic force to allow the switch valve 13 to abut against and seal a wall of the water inlet channel 12. The power transmission member 25 includes a moving rod 251 and an elastic deformation rod 252. The moving rod 251 is movably arranged on a side wall of the water inlet channel 12. The elastic deformation rod 252 is arranged inside the water inlet channel 12 and abuts against the switch valve 13. The moving lever 251 may receive a force applied by the control button 21 and is driven to move to the inside of the water inlet channel 12 to abut against the elastic deformation rod 252, and the elastic deformation rod 252 is bent and deformed. The elastic deformation rod 252 being bent and deformed may overcome an elastic force applied by the valve drive member 26 and may lift the switch valve 13, such that the opening of the water inlet channel 12 may be opened, allowing the water inlet port 241, the moving cavity, and the water inlet channel 12 to be communicated with each other, allowing the nasal cleansing solution to enter the water inlet channel 12. Reversely, when the control button 21 is released, the moving rod 251 and the elastic deformation rod 252 are reset to initial positions by an action of the valve drive member 26, such that the switch valve 13 closes the opening of the water inlet channel 12. More specifically, an inside of the control button 21 is arranged with an extension bar 211. An end of the extension bar 211 abuts against an end of the moving bar 251 protruding out of the water inlet channel 12. In this way, when the control button 21 is pressed, the extension bar 211 abuts against to move the moving bar 251, such that the water inlet channel 12 is opened.

Further, the control portion 20 further includes a suction trigger 27 aligned to and connected to the control button 21. The suction trigger 27 is received inside the central control housing 24 and disposed out of the water inlet channel 12 and the water return channel 22. The suction member 23 is received inside the central control housing 24 and disposed out of the water inlet channel 12 and the water return channel 22. The suction member 23 is electrically connected to the suction trigger 27. The suction trigger 27 is configured to control the suction member 23 to start operating or stop operating. While the control button 21 is controlling the switch valve 13 to be opened via the power transmission member 25, the power transmission member 25 is triggering the suction trigger 27 to control the suction member 23 to start operating.

In the present embodiment, the central control housing 24 further includes a bottom housing 245 opposite to the top housing 243. The bottom housing 245 covers a bottom of the side housing 242. The bottom housing 245 defines a bottom hole communicating with the waste water chamber 41, and the water return channel 22 is communicated with the bottom hole. A side of the bottom housing 245 facing the top housing 243 is arranged with a support 245. The support 245 fixedly supports the suction member 23 and the suction trigger 27. The extension rod 211 is connected to a trigger terminal of the suction trigger 27. When the control button 21 is pressed, the extension bar 211 moves to approach the suction trigger 27 and thus abuts against the trigger terminal of the suction trigger 27, such that the suction trigger 27 is enabled to trigger the suction member 23 to start operating. When the control button 21 is released, the extension bar 211 is separated from the suction trigger 27, and therefore, the suction member 23 stops operating.

Further, the water return portion 40 includes a return housing 42 and an intermediate cover 43. The waste water chamber 41 is arranged inside the return housing 42. The intermediate cover 43 closes the opening of the waste water chamber 41 facing towards the central control housing 24. The water return channel 22 extends through the intermediate cover 43. The suction member 23 is arranged with a suction pipe 231 that extends through the intermediate cover 43. The suction pipe 231 is communicated to the waste water chamber 41. The suction member 23 provides the negative pressure to the water return channel 22 via the waste water chamber 41.

In the present embodiment, the intermediate cover 43 abuts against a side of the bottom housing away from the top cover 243. The return housing 42 is detachable from the intermediate cover 43, allowing the waste water in the waste water chamber 41 to be discharged. The bottom hole penetrates through the intermediate cover 43. In this way, the waste water flowing out of the water return channel 22 may flow into the waste water chamber 41. The suction pipe 231 is arranged side by side with the water return channel 22. The suction pipe 231 passes through the bottom housing 245 and the intermediate cover 43, enabling the suction member 23 to be connected with the waste water chamber 41 and the return water channel 22. When the suction member 23 is operating, the negative pressure is generated in the waste water chamber 41, and therefore, the negative pressure is generated in the water return channel 22.

Further, the control portion 20 further includes a battery 28, a charging interface 29, and a circuit board 210. The battery 28 is fixedly received in the central control housing 24. The circuit board 210 is fixedly received in the central control housing 24 and electrically connected to the battery 28, the suction member 23, and the suction trigger 27. The charging interface 29 is disposed on a side of the central control housing 24 and electrically connected to the circuit board 210 to be connected to an external power supply and to charge the battery 28.

In the present embodiment, the charging port 29 is arranged on the side housing 242. The charging port 29 and the control button 21 are arranged on a same side of the side housing 242. The charging interface 29 is a TPYE-C interface, such that the charging interface 29 may be connected to a standard charging cable, allowing the battery 28 to receive electrical energy. The battery 28 is fixed to the bottom housing 245. The battery 28 is received inside the side housing 242 and disposed out of the water inlet channel 12 and the water return channel 22. The circuit board 210 is fixed to the support 245 and is disposed facing the charging interface 29, enabling the charging interface 29 to be connected to the circuit board 210 easily. A circuit module on the circuit board 210 may process the electrical energy obtained from the charging interface 29 and convert and transmit the electrical energy to the battery 28 to charge the battery 28. The circuit board 210 may process commands sent from the suction trigger 27 to control the suction member 23 to be turned on or to start operating. The battery 28 is a lithium battery 28. The battery 28 may be recharged and discharged for various times and ensure the automatic nasal washer 100 to have an extended operating duration.

Further, the control portion 20 further includes a status indicator light 220. The status indicator light 220 is arranged on a side of the central control housing 24 and electrically connected to the circuit board 210 to indicate a charging status of the battery 28.

In the present embodiment, the status indicator light 220 is arranged on the side housing 242 and is adjacent to the charging interface 29. The status indicator light 220 may display different colors depending on a remaining power of the battery 28. For example, while the battery 28 is discharging, the status indicator light 220 may emit blue light constantly when the remaining power of the battery 28 is greater than 20%. The status indicator light 220 may emit red light constantly when the remaining power of the battery 28 is less than 20%. The status indicator light 220 may emit the red light and flash when the remaining power of the battery 28 is nearly depleted. While the battery 28 is being charged, the status indicator light 220 may emit the red light and flash when the remaining power of the battery 28 is less than 20%, and the status indicator light 220 may emit blue light constantly when the remaining power of the battery 28 is greater than 20%. In other embodiments, the status indicator light 220 may further be configured to indicate a state of whether the control button 21 is being pressed. The status indicator light 220 may be on when the control button 21 is pressed. The status indicator light 220 may be off when the control button 21 is not pressed.

Further, the control portion 20 is arranged with a gyroscope 230 and a controller 240. The gyroscope 230 is configured to sense an attitude of the automatic nasal washer 100. The gyroscope 230 is electrically connected to the controller 240 to send the attitude of the automatic nasal washer 100 to the controller 240. The controller 240 is electrically connected to the suction member 23. The controller 240 is configured to control the suction member 23 to stop operating when the attitude of the automatic nasal washer 100 shows the automatic nasal washer 100 being inclined.

In the present embodiment, the gyroscope 230 is fixed to the circuit board 210 or is fixedly arranged inside the bottom housing 245. The gyroscope 230 is electrically connected to the circuit board 210. The controller 240 is arranged on the circuit board 210 to process information sensed by the gyroscope 230. When the user presses the control button 21, and when the gyroscope 230 senses that the automatic nasal washer 100 is tilted at an angle of more than 30° with respect to a vertical direction, the controller 240 controls the suction member 23 to stop operating. In this way, the suction trigger 27 fails to control the operation of the suction member 23, preventing the nasal docking portion 30 of the automatic nasal washer 100 from outputting water while the automatic nasal washer 100 is not in use, ensuring safety of the automatic nasal washer 100.

Further, the nasal docking portion 30 is arranged with a first port 301 and a second port 302 disposed side by side with the first port 301. An end of the first port 301 is communicated with the water inlet channel 12, and an end of the second port 302 is communicated with the water return channel 22. The other end of the first port 301 and the other end of the second port 302 are communicated with two nostrils of the user respectively.

In the present embodiment, the nasal docking portion 30 has an outer end 303 and an inner end 304 opposite to the outer end 303. The outer end 303 is disposed outside the side housing 242. A portion of the first port 301 communicating with the nostril of the user and a portion of the second port 302 communicating with the nostril of the user are arranged at the outer end 303. The inner end 304 is arranged inside the side housing 242. The first port 301 is communicated with the water inlet water channel 12 at the inner end 304. The second port 302 is communicated with the water return channel 22 at the inner end 304. The inner end 304 is sealed to both the water inlet channel 12 and the water return channel 22 to ensure the water in the water inlet channel 12 to be output through the first port 301 and to ensure the second port 302 to receive and transfer the waste water flowing from the nasal cavity to the water return channel 22.

Further, the nasal docking portion 30 is arranged with a diversion head 305 communicated with the water inlet channel 12 and the water return channel 22. The diversion head 305 is rotatably arranged on the control portion 20 to change the water flowing direction towards to the first port 301 or the second port 302.

In the present embodiment, the diversion head 305 is arranged at the inner end 304. The diversion head 305 includes an intermediate channel 306 and a peripheral channel 307. The first port 301 is communicated with an end of the intermediate channel 306. The second port 302 is communicated with an end of the peripheral channel 307. The other end of the intermediate channel 306 is communicated with the water inlet channel 12. The other end of the peripheral channel 307 is communicated with the water return channel 22. The diversion head 305 is rotatable around an axis of the intermediate channel 306 to allow the intermediate channel 306 to be always connected to the water inlet channel 12 and to allow the peripheral channel 307 to be always connected to the water return channel 22. After the diversion head 305 is rotated for 180°, a position of the outer end of the first port 301 and a position of the outer end of the second port 302 can be interchanged, such that a direction of water in and out of the left nostril and a direction of water in and out of the right nostril can be switched by the user easily, improving user experience.

For the automatic nasal washer 100 in the present disclosure, the switch valve 13 may be controlled by the control portion 20 to be opened or closed, such that the nasal docking portion 30 may automatically output cleansing water to the nasal cavity of the user, and the nasal docking portion 30 may automatically withdraw the waste water flowing from the nasal cavity of the user. Operations performed by the user may be simple and quickly.

In summary, although the present disclosure is disclosed in the above by providing preferred embodiments, the preferred embodiments are not intended to limit the present disclosure. Any ordinary skilled person in the art may perform various changes and embellishments without departing from the concept and scope of the present disclosure. Therefore, the scope of the present disclosure is subject to the scope defined by the claims.

What is claimed is:

1. An automatic nasal washer, comprising a water storage portion, a control portion, and a nasal docking portion,
wherein the water storage portion is arranged with a water storage chamber;
the control portion is fixed to the water storage portion and is arranged with: a water inlet channel communicated with the water storage chamber; a switch valve configured to control the water inlet channel to be communicated with or dis-communicated from the water storage chamber; a water return channel communicated with the water inlet channel; and a suction member configured to provide a negative pressure to the water return channel;
the nasal docking portion is connected to a point where the water inlet channel is connected to the water return channel; and is configured to output washing water from the water inlet channel to a nasal cavity of a user and to withdraw waste water flowing from the nasal cavity to the water return channel.

2. The automatic nasal washer according to claim 1, wherein the water storage portion comprises a storage housing, a filter housing, and a cover; the water storage chamber is defined in the storage housing, the water storage chamber has a top opening and a bottom opening, the cover closes the top opening; the filter housing is received inside the water storage chamber and is connected to the top opening; the control portion is connected to the bottom opening, and the switch valve is disposed at the bottom opening.

3. The automatic nasal washer according to claim 2, wherein the control portion comprises a central control housing, a power transmission member, a valve drive member, and a control button;
the central control housing is sealed to the bottom opening and defines a water inlet port communicated with the bottom opening; the switch valve is disposed at the water inlet port; the water inlet channel and the water return channel are arranged inside the central control housing;
the control button is configured to control the switch valve to be opened or closed; the control button is pressable and arranged on a side of the central control housing; the valve drive member is connected to the switch valve to provide an abutting force to close the valve; the power transmission member is connected to the control button and the valve drive member to transfer a pressing force of the control button to the valve drive member to control the valve drive member to open the switch valve.

4. The automatic nasal washer according to claim 3, wherein the control portion further comprises a suction trigger connected with the control button;
the suction trigger is received inside the central control housing and is disposed out of the water inlet channel and the water return channel; the suction member is received in the central control housing and is disposed out of the water inlet channel and the water return channel, the suction member is electrically connected to the suction trigger; the suction trigger is configured to control the suction member to start operating or stop operating; and while the control button is controlling the switch valve to be opened via the power transmission member, the suction trigger is being triggered to control the suction member to start operating.

5. The automatic nasal washer according to claim 4, further comprising a water return portion, wherein the water return section is fixed on a side of the control portion away from the water storage portion and defines a waste water chamber communicated with the water return channel, the waste water chamber is configured to receive the waste water flowing from the water return channel.

6. The automatic nasal washer according to claim 4, wherein the control portion further comprises a battery, a charging interface, and a circuit board; the battery is fixedly arranged inside the central control housing; the circuit board is fixedly arranged inside the central control housing and electrically connected to the battery, the suction member, and the suction trigger; the charging interface is arranged on a side of the central control housing and electrically connected to the circuit board to allow an external power supply to charge the battery.

7. The automatic nasal washer according to claim 6, wherein the control portion further comprises a status indicator light, the status indicator light is arranged on a side of the central control housing and electrically connected to the circuit board to indicate a charging state of the battery.

8. The automatic nasal washer according to claim 1, wherein the control portion is arranged with a gyroscope and a controller; the gyroscope is configured to sense an attitude of the automatic nasal washer and is electrically connected to the controller to send the attitude of the automatic nasal washer to the controller; the controller is electrically connected to the suction member and is configured to control the suction member to stop operating in response to the attitude of the automatic nasal washer being inclined.

9. The automatic nasal washer according to claim 1, wherein the nasal docking portion is arranged with a first port and a second port side by side with the first port; an end of the first port is connected to the water inlet channel, an end of the second port is connected to the water return channel; the other end of the first port and the other end of the second port are connected to two nostrils of the user, respectively.

10. The automatic nasal washer according to claim 9, wherein the nasal docking portion is arranged with a diversion head communicated with the water inlet channel and the water return channel; and the diversion head is rotatably arranged on the control portion to change a direction of the first port and the second port.
